(19) United States
(12) Patent Application Publication
(10) Pub. No.: US 2009/0131523 A1
(43) Pub. Date: May 21, 2009

(54) Lipid compositions for the treatment and prevention of proliferative diseases and for the reduction of incidences of mutagenesis and carinogenesis

(75) Inventor: Fabiana Bar Yosef, Haifa (IL)

Correspondence Address: THE NATH LAW GROUP, 112 South West Street, Alexandria, VA, 22314, US

(73) Assignee: Enzymotec Ltd., Migdal Haemek (IL)

(21) Appl. No.: 12/285,806

(22) Filed: Oct. 14, 2008

Related U.S. Application Data
(60) Provisional application No. 60/960,798, filed on Oct. 15, 2007.

(51) Int. Cl. A61K 31/20; A23D 7/005; A23D 7/04; A23L 1/29

(52) U.S. Cl. 514/558; 426/2

(57) ABSTRACT

Lipid compositions are provided for the treatment and prevention of proliferative diseases and for the reduction of incidences of mutagenesis and carcinogenesis.

This application claims the benefit of prior U.S. provisional patent application No. 60/960,798 filed Oct. 15, 2007, the contents of which are hereby incorporated by reference in their entirety.

### FIELD OF THE INVENTION

This invention relates to the field of reduction of incidences of mutagenesis and carcinogenesis and treatment and prevention of proliferative diseases and disorders.

### BACKGROUND OF THE INVENTION

Animal and human studies have shown that a high level of dietary fat reduces the time between UV or other types of radiation exposure and tumor appearance, increases the number of tumors, and affects the promotional stage of UV carcinogenesis. Other studies have shown that higher dietary intake of ω-3 and ω-6 fatty acids may reduce the risk of several types of carcinomas. The fatty acid profile of the erythrocyte membrane reflects dietary macronutrient intake and the interactions between dietary intake and endocrine changes.

Hardy et al. [Cancer Research 60, 6353-6358 (2000)] have shown the effects of free fatty acids, oleate and palmitate on established human breast cancer cell lines. Oleate was shown to stimulate cell proliferation, whereas palmitate was shown to inhibit cell proliferation.

Nadathur S R et al. [Mutation Research 359, 179-189 (1996)], showed that palmitic acid in yoghurt exerts anti-N-methyl-N′-nitro-N-nitrosoguanidine (MNNG) activity.

Harris et al. [Cancer Epidemiol Biomarkers Prev 14(4), 906-12 (2005)] showed that increasing proportions of dietary palmitic acid and palmitoleic acid were associated with reduced squamous cell carcinoma (SCC).

Eitel K. et al. [Diabetes 52, 991-997 (2003)] have shown a proapoptotic effect of PKC-δ that is induced by the saturated fatty acids palmitate and stearate.

### SUMMARY OF THE INVENTION

The present invention provides a use of a lipid composition comprising at least one triglyceride of the following formula I:

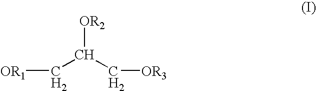

wherein R₁, R₂ and R₃ may be identical or different and are each independently selected from H or an acyl group, wherein said acyl group is selected from a group consisting of saturated, mono-unsaturated and poly-unsaturated fatty acid residues, and wherein the total palmitic acid residue content is from about 15% to about 55% of the total fatty acid residues in the composition, for the preparation of a nutritional, pharmaceutical or nutraceutical composition or a functional food, for the reduction of incidences of mutagenesis and carcinogenesis and for the prevention and treatment of proliferative diseases and disorders.

The subject invention further envisages a method of treating a subject having, or having an increased risk of incidences of mutagenesis, carcinogenesis and proliferative disease or disorder comprising administering to the subject a lipid composition comprising at least one triglyceride of the following formula I:

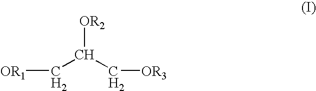

wherein R₁, R₂ and R₃ may be identical or different and are each independently selected from H or an acyl group, wherein said acyl group is selected from a group consisting of saturated, mono-unsaturated and poly-unsaturated fatty acid residues and wherein the total palmitic acid residue content is from about 15% to about 55% of the total fatty acid residues in the composition.

### DRAWINGS OF THE INVENTION

In order to understand the invention and to see how it may be carried out in practice, embodiments will now be described, by way of non-limiting example only, with reference to the accompanying drawings, in which:

FIGS. 1A-1C show the effect of Fat blend 6 (oil enriched with a high content of palmitic acid at the sn-2 position) and a oil containing a low content of palmitic acid (LPO) on the proliferation rate of human peripheral blood mononuclear cells (huPBMCs). FIG. 1A demonstrates the proliferation rate of huPBMCs cells when either Fat blend 6 or LPO are applied simultaneously with PHA (phytohemagglutinin, Lectin from Phaseolus vulgaris (red kidney bean)), proliferation activator. FIG. 1B demonstrates the proliferation rate of huPBMCs cells when Fat blend 6 and LPO are applied one day prior to PHA application; and FIG. 1C demonstrates the proliferation rate of huPBMCs cells when Fat blend 6 and LPO are applied one day after PHA application.

FIG. 2 shows the effect of tested oils, Fat blend 6 (oil enriched with a high content of palmitic acid at the sn-2 position) and LPO (oil containing a low content of palmitic acid) on mutation rate occurrence in Mouse Lymphoma Assay (MLA) in comparison to a negative and positive control.

FIG. 3 shows the effect of Fat blend 6 (oil enriched with a high content of palmitic acid at the sn-2 position) and LPO (oil containing a low content of palmitic acid) on a large/small colony sizing ratio of mouse lymphoma cells in comparison to a negative and positive control.

### DETAILED DESCRIPTION OF THE INVENTION

The present invention provides a use of a lipid composition comprising at least one triglyceride of the following formula I:

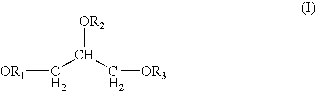

wherein R₁, R₂ and R₃ may be identical or different and are each independently selected from H or an acyl group, wherein said acyl group is selected from a group consisting of saturated, mono-unsaturated and poly-unsaturated fatty acid residues, and wherein the total palmitic acid residue content is from about 15% to about 55% of the total fatty acid residues in the composition, for the preparation of a nutritional, pharmaceutical or nutraceutical composition or a functional food, for the reduction of incidences of mutagenesis and carcinogenesis and for the prevention and treatment of proliferative diseases and disorders.

The lipid composition used in the invention typically comprises a mixture of said triglycerides of formula I. Such a mixture comprises two or more triglycerides of formula I.

The present invention further envisages a method of treating a subject having, or having an increased risk of incidences of mutagenesis, carcinogenesis and proliferative disease or disorder comprising administering to the subject a lipid composition comprising at least one triglyceride of the following formula I:

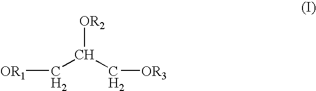

wherein R₁, R₂ and R₃ may be identical or different and are each independently selected from H or an acyl group, wherein said acyl group is selected from a group consisting of saturated, mono-unsaturated and poly-unsaturated fatty acid residues and wherein the total palmitic acid residue content is from about 15% to about 55% of the total fatty acid residues in the composition.

The present invention further envisages a method of preventing incidences of mutagenesis, carcinogenesis and proliferative disease or disorder in a subject comprising administering to the subject a lipid composition comprising at least one triglyceride of the following formula I:

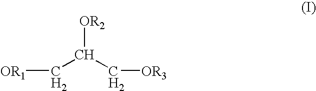

wherein R₁, R₂ and R₃ may be identical or different and are each independently selected from H or an acyl group, wherein said acyl group is selected from a group consisting of saturated, mono-unsaturated and poly-unsaturated fatty acid residues and wherein the total palmitic acid residue content is from about 15% to about 55% of the total fatty acid residues in the composition.

Compositions of the subject invention are intended for reducing incidences of mutagenesis and carcinogenesis and for treating or preventing proliferative diseases and disorders in a subject.

The term “subject” as used herein should be understood to encompass any mammal, including, but not limited to, humans, household animals (e.g. cat, dog), and farm animals (e.g. cow, goat, ship). Said subject may be in any stage of development or maturity in its life cycle. The subject can be, but is not limited to, a newborn, a preterm and term infant, a toddler, a child, an adolescent, an adult or a geriatric subject.

A composition as used herein can be, but is not limited to, a nutritional composition (such as infant formula), a nutraceutical composition (such as a dietary supplement), a functional food, a medical food or a pharmaceutical composition.

In one embodiment, a lipid composition for use in the invention is prepared from a natural, synthetic or semi-synthetic source. In a further specific embodiment, said natural source is any one of plant, animal or microorganism source. In yet a further embodiment, the production of said lipid composition involves an enzymatic catalysis.

A nutritional composition as used herein can be any nutritional composition including, but not limited to, human milk fat substitute, infant formula, dairy product, ice-cream, biscuit, soy product, bakery, pastry and bread, sauce, soup, prepared food, frozen food, condiment, confectionary, oils and fat, margarine, spread, filling, cereal, instant product, infant food, toddler food, bar, snack, candy and chocolate product.

A functional food as used herein can be any functional food, including, but not limited to, dairy product, ice-cream, biscuit, soy product, bakery, pastry and bread, sauce, soup, prepared food, frozen food, condiment, confectionary, oils and fat, margarine, spread, filling, cereal, instant product, drinks and shake, infant food, bar, snack, candy and chocolate product.

A nutraceutical composition as used herein can be any nutraceutical, which can be any substance that may be considered a food or part of a food and provides medical or health benefits, including the prevention and treatment of disease. Such nutraceutical compositions include, but are not limited to, a food additive, a food supplement, a dietary supplement, genetically engineered foods such as for example vegetables, herbal products, and processed foods such as cereals, soups and beverages and stimulant functional food, pharmafood and medical food.

In an embodiment of the invention, the pharmaceutical or nutraceutical compositions are in a dosage delivery form.

Suitable routes of administration for the compositions of the subject invention are oral, buccal, sublingual, via feeding tube, topical, transdermal, or parenteral (including subcutaneous, intramuscular, intravenous and intradermal) administration. In a specific embodiment, the compounds can be administered orally.

The exact dose and regimen of administration of the composition will necessarily be dependent upon the therapeutic effect to be achieved (e.g. treatment of proliferative disease) and may vary with the particular formula, the route of administration, and the age and condition of the individual subject to whom the composition is to be administered.

The present invention thus also provides pharmaceutical compositions for use in the invention in admixture with pharmaceutically acceptable auxiliaries, and optionally other therapeutic agents. The auxiliaries must be “acceptable” in the sense of being compatible with the other ingredients of the composition and not deleterious to the recipients thereof.

In one embodiment, the pharmaceutical composition further comprises at least one pharmaceutically active agent.

The compositions may be prepared by any method well known in the art of pharmacy. Such methods include the step of bringing in association the ingredients with any auxiliary agent. The auxiliary agent(s), also named accessory ingredient(s), include those conventional in the art, such as carriers, fillers, binders, diluents, disintegrants, lubricants, colorants, flavouring agents, anti-oxidants, and wetting agents.

Pharmaceutical compositions suitable for oral administration may be presented as discrete dosage units such as pills, tablets, dragées or capsules, or as a powder or granules, or as a solution or suspension.

For parenteral administration, suitable compositions include aqueous and non-aqueous sterile injection. The compositions may be presented in unit-dose or multi-dose containers, for example sealed vials and ampoules, and may be stored in a freeze-dried (lyophilised) condition requiring only the addition of sterile liquid carrier, for example water, prior to use. For transdermal administration, e.g. gels, patches or sprays can be contemplated.

The invention further provides a commercial package for preparing a composition for use in the invention such as an edible fat source or food article in accordance with the invention comprising (a) a fat source which upon administration to a subject prevents or treats a proliferative disorder or disease and/or reduces incidences of mutagenesis and carcinogenesis, (b) optionally at least one of edible physiologically acceptable protein, carbohydrate, vitamin, mineral, amino acid, nucleotide and active or non-active additive; (c) optionally at least one edible physiologically acceptable carrier or diluent for carrying the constituent/s defined in (a) and (b); (d) means and receptacles for admixing the constituents defined in (a), (b) and/or (c); and (e) instructions for use such as, but not limited to terms of storage, instructions for preparation of the fat source or food article for administration, required dilutions, dosages, frequency of administration and the like.

A commercial package in accordance with the invention may also contain a fat source of the invention in a ready-to-use form, together with instructions for use. Dosages are usually determined according to age, weight, sex and condition of the subject, in accordance with good medical practice known to the attending physician and other medical personnel.

As used herein, the term “mutagenesis” relates to a process which produces and results in mutations; this may lead to transformation and carcinogenesis. Two classes of gene mutations are recognized: point mutations and intragenic deletions. Point mutations involve substitution, addition or deletion of one or a few DNA bases from a polynucleotide sequence. These mutations are all called sign mutations or frame-shift mutations because of their effect on the translation of the information of the gene. More extensive deletions can occur within the gene, which are sometimes difficult to distinguish from mutants which involve only one or two bases. In the most extreme case, all the informational material of the gene is lost.

As used herein the term “carcinogenesis” relates to a process which leads to development of cancer. Carcinogenesis may be a matter of induction (by chemical, physical, or biological agents) of neoplasms that are usually not observed, an earlier induction of neoplasms than are usually observed, and/or the induction of more neoplasms than are usually found. Carcinogenesis is a result of a mutagenenic occurrence.

As used herein, the term “proliferative disease” or “proliferative disorder” is intended to mean a disease that is caused by or results in inappropriately high levels of cell division, inappropriately low levels of apoptosis, or both. Non limiting examples of proliferative disease or disorders include cancer, autoimmune disorders, anti-inflammatory diseases, cutaneous lymphoproliferative diseases, rheumatic diseases, and so forth.

As used herein, the term “reduction of incidences of mutagenesis and/or carcinogenesis” refers to amelioration, prevention, slowing down the progression of mutagenesis and carcinogenesis, slowing down the deterioration caused by mutagenesis and carcinogenesis, prolonging the time period for onset of remission of diseases associated with mutagenesis and carcinogenesis, slowing down irreversible damage caused by a progressive chronic stage of diseases caused by mutagenesis and carcinogenesis, delaying the onset of diseases associated with mutagenesis and carcinogenesis, lessening the severity of diseases associated with mutagenesis and carcinogenesis, improving the survival rates of individuals suffering from diseases associated with mutagenesis and carcinogenesis, or any combination of the above.

As used herein, the term “cancer” should be understood to encompass a disease state in which a carcinogenic agent or agents cause the transformation of a healthy cell into an abnormal cell, which may be followed by an invasion of adjacent tissues by these abnormal cells, and which may be followed by lymphatic, cerebral spinal fluid, or blood-borne spread of these abnormal cells to regional lymph nodes and/or distant sites, i.e. metastasis.

The term “cancer” as used herein should further be understood to encompass any neoplastic disease (whether invasive or metastatic) which is characterized by abnormal and uncontrolled cell division causing malignant growth or tumor. Non-limiting examples of cancer which may be treated with a composition of the invention are leukemia, melanoma, breast cancer, pancreatic cancer, lung cancer, prostate cancer, colorectal and/or colon cancer, hepatocellular carcinoma, lymphoma (including non-Hodgkin's lymphoma and mycosis fungoides), sarcoma, mesothelioma, brain cancer (including glioma), germinoma (including testicular cancer and ovarian cancer), choriocarcinoma, renal cancer, thyroid cancer, head and neck cancer, endometrial cancer, cervical cancer, bladder cancer, or stomach cancer, glioma, anaplastic astrocytoma, glioblastoma multiforme and so forth.

In another embodiment, a method of the invention further comprises administering to the subject in need of treatment or prevention of proliferative disease or disorder and reduction of incidences of mutagenesis and carcinogenesis, at least one additional drug in addition to a lipid composition as used in the subject invention.

Such additional drug may be administered to the subject in conjunction with, whether concomitant or sequential, administration of a lipid composition of the invention. Non limiting examples of additional drugs are anti-proliferative drugs, anti-inflammatory drugs, drugs which are know to increase the risk of said subject to proliferative diseases, and any treatment intended to inhibit the replication of cancer cells, to inhibit the spread of cancer, to decrease tumor size, and to lessen or to reduce the number of cancerous cells in the body.

In another embodiment, a method of the invention further comprises subjecting a subject to at least one additional anti-proliferative treatment. Such treatment may be performed in conjunction with, whether concomitant or sequential, administration of a lipid composition of the invention and optionally also said at least one additional drug. Non limiting examples of anti-proliferative treatment are chemotherapy, radiation therapy, surgery, and any treatment intended to inhibit the replication of cancer cells, to inhibit the spread of cancer, to decrease tumor size, or to lessen or to reduce the number of cancerous cells in the body.

A fat blend is used in the preparation of a lipid composition for use in the subject invention. Such composition may further comprise other components such as, but not limited to a protein source, a carbohydrate source, minerals, vitamins, nucleotides, amino acids and optionally at least one of a carrier, diluent, additive or excipient, all of which are edible.

A fat blend as used herein for the preparation of a lipid composition for use in the invention, can be, but is not limited to, any fat source such as those described in WO05/036987 which include fat concentrates, also named fat bases. The fat blend used in the present invention can be any dietary ingredient comprising an edible fat source. In a particular embodiment, fat blends are those which are based on synthetic triglycerides (which can be produced both chemically and enzymatically), which mimic the triglyceride composition of human breast milk fat. In one embodiment, such fat base (and fat blend) contains a high level of palmitic acid at the sn-2 position of the triglycerides of Formula I (e.g., at least 30%), and a high level of unsaturated fatty acids at sn-1 and sn-3 positions of the triglycerides of Formula I (e.g. at least 50% of the total fatty acid at sn-1 and sn-3 positions). A non-limiting example is a blend named InFat® (Enzymotec Ltd., Migdal HaEmeq, Israel).

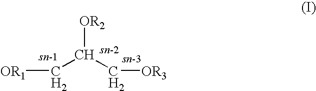

Since fat blends are prepared by blending fat base with other oils, the fatty acid composition of the fat blends results from the fatty acid composition of both the fat base and the other oils mixed with the fat base.

A composition as used in the subject invention can be a substitute human milk fat composition comprising a fat blend consisting of at least 25% of a fat base with up to 75% of at least one vegetable oil.

Non-limiting examples of vegetable oil used in the preparation of blends used in the invention are soy, palm tree, canola, coconut, palm kernel, sunflower, corn and rapeseed oil, as well as other vegetable oils and fats and mixtures thereof.

As used herein, the term “lipid” related to fats and fatlike compounds, which are essentially insoluble in water and which include, but are not limited to, triglycerides, sterols, fatty acids, and so forth.

As used herein, the term “acyl group” relates to an organic radical denoted —C(═O)R, wherein R is selected from saturated, mono-unsaturated and polyunsaturated C₄-C₂₈ aliphatic residue of the fatty acid residue.

As used herein, the term ‘fatty acid’ relates to a carboxylic acid with a long unbranched aliphatic tail (chain), which is either saturated or unsaturated having one unsaturated bond (mono-unsaturated fatty acids) or two or more unsaturated bonds (poly-unsaturated fatty acids).

Non-limiting examples of saturated fatty acids which may be used in this invention include: Butyric acid (Butanoic acid, C4:0), Caproicacid (Hexanoic acid, C6:0), Caprylic acid (Octanoic acid, C8:0), Capric acid (Decanoic acid, C10:0), Lauric acid (Dodecanoic acid, C12:0), Myristic acid (Tetradecanoic acid, C14:0), Palmitic acid (Hexadecanoic acid, C16:0), Stearic acid (Octadecanoic acid, C18:0), Arachidicaicd (Eicosanoic acid, C20:0), Behenic acid (Docosanoic acid, C22:0).

Non-limiting examples of unsaturated fatty acids which may be used in this invention include: Myristoleic acid (ω-5, C14:1), Palmitoleic acid (ω-7, C16:1), Oleic acid (ω-9, C18:1), Linoleic acid (ω-6, C18:2), Alpha-linolenic acid (ω-3, C18:3), Arachidonic acid (ω-6, C20:4), Eicosapentaenoic acid (ω-3, C20:5), Erucic acid (ω-9, C22:1) and Docosahexaenoic acid (ω-3, C22:6).

In one embodiment of the invention, R₂ is a saturated fatty acid residue. In a further embodiment, the saturated fatty residue is selected from C₁₄-C₁₈ saturated fatty acid residues. In yet a further embodiment, the saturated fatty acid is a palmitic acid residue.

In one embodiment, the total palmitic acid residue content is from about 15% to about 40% of the total fatty acid residues in the composition. In another embodiment, the total palmitic acid residue content is from about 15% to about 33% of the total fatty acid residues in the composition.

In another embodiment of the invention, R₁ and R₃ are both H.

In one embodiment, at least 13% of the total fatty acids at the sn-2 position of the triglyceride backbone are palmitic acid residues. In another embodiment, at least 15% of the total fatty acids at the sn-2 position of the triglyceride backbone are palmitic acid residues. In yet another embodiment, at least 18% of the total fatty acids at the sn-2 position of the triglyceride backbone are palmitic acid residues. In yet another embodiment, at least 22% of the total fatty acids at the sn-2 position of the triglyceride backbone are palmitic acid residues.

In one embodiment, at least 30% of the total palmitic acid residues in the composition are bonded at the sn-2 position of the triglyceride backbone. In another embodiment, at least 33% of the total palmitic acid residues are bonded at the sn-2 position of the triglyceride backbone. In yet another embodiment, at least 38% of the total palmitic acid residues are bonded at the sn-2 position of the triglyceride backbone. In yet another embodiment, at least 40% of the total palmitic acid residues are bonded at the sn-2 position of the triglyceride backbone.

In a further embodiment of the invention, R₁ and R₃ are unsaturated fatty acid residues.

In one embodiment, at least 50% of the total fatty acid residues at the sn-1 and sn-3 positions of the triglyceride backbone are unsaturated. In a further embodiment, at least 70% of the total fatty acid residues at the sn-1 and sn-3 positions of the triglyceride backbone are unsaturated. In one embodiment, said unsaturated fatty acid residue is selected from the group consisting of oleic acid, linoleic acid, linolenic acid and gadoleic acid. In one specific embodiment, at least 35% of the unsaturated fatty acid residues at the sn-1 and sn-3 positions are oleic acid residues. In a further specific embodiment, at least 40% of the unsaturated fatty acid residues at the sn-1 and sn-3 positions are oleic acid residues.

In one specific embodiment, at least 4% of said unsaturated fatty acid residues at the sn-1 and sn-3 positions are linoleic acid residues. In a further specific embodiment, at least 6% of said unsaturated fatty acid residues at the sn-1 and sn-3 positions are linoleic acid residues.

In a first embodiment, said lipid composition consists of:
0-10% C8:0 fatty acids out of the total fatty acids;
0-10% C10:0 fatty acids out of the total fatty acids;
0-22% C12:0 fatty acids out of the total fatty acids;
0-15% C14:0 fatty acids out of the total fatty acids;
15-55% C16:0 fatty acids out of the total fatty acids; wherein at least 30% at sn-2 position;
1-7% C18:0 fatty acids out of the total fatty acids;
20-75% C18:1 fatty acids out of the total fatty acids;
2-40% C18:2 fatty acids out of the total fatty acids;
0-8% C18:3 fatty acids out of the total fatty acids;
other fatty acids are present in levels of less than 8% of the total fatty acids.

In a second embodiment, said lipid composition consists of:
5-15% C12:0 fatty acids out of the total fatty acids;
2-10% C14:0 fatty acids out of the total fatty acids;
17-25% C16:0 fatty acids out of the total fatty acids; wherein at least 40% at sn-2 position;
2-5% C18:0 fatty acids out of the total fatty acids;
28-45% C18:1 fatty acids out of the total fatty acids;
5-20% C18:2 fatty acids out of the total fatty acids;
1-3% C18:3 fatty acids out of the total fatty acids;
other fatty acids are present in levels of less than 5% of the total fatty acids.

All possible combinations of said first and said second embodiments are also envisaged. For example:
0-22% C12:0 fatty acids out of the total fatty acids, (from the first embodiment) can be combined with
2-10% C14:0 fatty acids out of the total fatty acids;
20-25% C16:0 fatty acids out of the total fatty acids;
2-5% C18:0 fatty acids out of the total fatty acids;
28-45% C18:1 fatty acids out of the total fatty acids;
5-20% C18:2 fatty acids out of the total fatty acids;
1-3% C18:3 fatty acids out of the total fatty acids; other fatty acids are present in levels of less than 5% of the total fatty acids.

It is to be understood that the compounds provided herein may contain chiral centers. Such chiral centers may be of either the (R) or (S) configuration, or may be any mixture thereof. Thus, the compounds provided herein may be enantiomerically pure, or be stereoisomeric or diastereomeric mixtures such as racemic or non-racemic mixtures.

### EXAMPLES

The invention is further described in the following examples, which are not in any way intended to limit the scope of the inventions as claimed.

### Example 1

### Compositions

Table 1 details the contents of several fat bases (hereinafter “fat bases”). Table 2 details the contents of several fat sources (hereinafter “Fat blends”) comprising either fat base 1, 7, 8, 9, 10 or 11 for use in the subject invention.

The fat base may represent about 30% up to about 83% of the Fat blends suitable for use in a composition for use in the invention.

The preparation of these fat sources is essentially as described in WO05/036987.

TABLE 1

| | fat | fat | fat | fat | fat | fat | fat | fat | fat | fat | fat |
|---|---|---|---|---|---|---|---|---|---|---|---|
| | base 1 | base 2 | base 3 | base 4 | base 5 | base 6 | base 7 | base 8 | base 9 | base 10 | base 11 |
| C12:0 | | | | | | | | | | | |
| C14:0 | | | | | | | | | | | |
| C16:0 | 32 | 29.4 | 29.6 | 32.6 | 32.2 | 30.6 | 29 | 29 | 30 | 33 | 30 |
| C16:0 at sn-2 | 67.2 | 59.7 | 61.3 | 66.1 | 66 | 62.9 | 55.6 | 53.9 | 59 | 52.9 | 55.8 |
| of total-fatty | | | | | | | | | | | |
| acids at sn-2 | | | | | | | | | | | |
| Ratio (%) of | 70.0 | 67.7 | 69.0 | 67.6 | 68.3 | 68.5 | 64 | 62 | 64 | 53.5 | 62 |
| sn-2 palmitic | | | | | | | | | | | |
| acid of total | | | | | | | | | | | |
| palmitic acid | | | | | | | | | | | |
| C18:0 | 4 | 4.4 | 4.4 | 4 | 4.1 | 3.8 | 2.6 | 2.6 | 3 | 3 | 3 |
| C18:1 | 53.1 | 55.9 | 55.5 | 53.1 | 53.4 | 55 | 56 | 55.5 | 56.1 | 52 | 56.1 |
| C18:2 | 8 | 7.8 | 8.2 | 8 | 7.9 | 8.3 | 9 | 9 | 8.5 | 10 | 8.5 |

All numbers represent % (w/w), except the ratio which is defined as %. C16:0 represents the total palmitic acid content. C16:0 at the sn-2 represents the % palmitic acid at sn-2 of total sn-2 positioned fatty acids. The ratio means the % of sn-2 palmitic acid of total palmitic acid {(% of sn-2 palmitic of total sn-2 positioned fatty acids)/3)/(% total palmitic acid)} × 100.

TABLE 2

| | | | | | | | | | |
|---|---|---|---|---|---|---|---|---|---|
| Prepa- | Prepa- | Prepa- | Prepa- | Prepa- | Prepa- | Prepa- | Prepa- | Prepa- | Prepa- |
| ration A | ration B | ration C | ration D | ration E | ration F | ration G | ration H | ration I | ration J |
| Fat | Fat | Fat | Fat | Fat | Fat | Fat | Fat | Fat | Fat |
| blend 1 | blend 2 | blend 3 | blend 4 | blend 5 | blend 6 | blend 7 | blend 8 | blend 9 | blend 10 |

Fatty acids

| | | | | | | | | | | |
|---|---|---|---|---|---|---|---|---|---|---|
| C12:0 | 11.1 | 7.2 | 7.8 | 6.5 | 4.4 | 8.7 | 8.1 | 13.4 | 10.1 | 10 |
| C14:0 | 4.5 | 3.1 | 3.3 | 2.8 | 2.1 | 3.5 | 2.9 | 5.3 | 3.7 | 4.2 |
| C16:0 | 22.8 | 25.4 | 26.9 | 25.1 | 27.7 | 21 | 21.6 | 15 | 22.1 | 17 |
| C16:0 at sn-2 | 33.4 | 42.9 | 48.9 | 50.8 | 56.9 | 31.8 | 31.3 | 25 | 28.7 | 16 |
| of total fatty | | | | | | | | | | |
| acids at sn-2 | | | | | | | | | | |
| Ratio (%) of | 48.7 | 56.3 | 60.7 | 67.4 | 68.5 | 50.5 | 48.3 | 55 | 43.3 | 31.5 |
| sn-2 palmitic | | | | | | | | | | |
| acid of total | | | | | | | | | | |
| palmitic acid | | | | | | | | | | |
| C18:0 | 2.3 | 3.0 | 3.1 | 3.5 | 4.0 | 2.6 | 2.6 | 2.9 | 2.7 | 3.2 |
| C18:1 | 38.4 | 40.8 | 41.6 | 47.9 | 46.6 | 44.4 | 42.7 | 39.7 | 43.9 | 41.7 |
| C18:2 | 13.5 | 15.6 | 12.8 | 8.6 | 11.7 | 16.4 | 18 | 15.3 | 13.6 | 18.2 |
| C18:3 | 1.7 | 0.6 | | 1.4 | | 1.5 | 1.8 | 2 | 1.4 | 2.1 |
| % Fat base 1 | 30 | 50 | 63 | 73 | 83 | | | | | |
| in fat blend | | | | | | | | | | |
| % Fat base 7 | | | | | | 60 | | | | |
| in fat blend | | | | | | | | | | |
| % Fat base 8 | | | | | | | 60 | | | |
| in fat blend | | | | | | | | | | |
| Fat base 9 in | | | | | | | | 36 | | |
| fat blend | | | | | | | | | | |
| Fat base 10 in | | | | | | | | | 52 | |
| fat blend | | | | | | | | | | |
| Fat base 11 in | | | | | | | | | | 25 |
| fat blend | | | | | | | | | | |

Vegetable oils

| | | | | | | | | | | |
|---|---|---|---|---|---|---|---|---|---|---|
| Palm kernel oil | | | | | | | 18 | | | |
| Coconut oil | 23 | 15 | 16 | 13.5 | 9.3 | 17 | | 28 | 21 | 21 |
| Palm oil | 21 | 15 | 9 | | | | | | | 14 |
| Sunflower | | 5 | | | 7.7 | | | 11 | | 14 |
| Corn oil | 10 | 10 | 12 | | | | | | 11 | |
| Safflower | | | | | | | | 3 | | 5 |
| Rapeseed | 16 | 5 | | 13.5 | | 6 | 4 | 20 | 16 | 21 |
| Soybean | | | | | | 17 | 18 | | | |
| Total | 100 | 100 | 100 | 100 | 100 | | 100 | 100 | 100 | 100 |

C16:0 represents the total palmitic acid content. C16:0 at the sn-2 represents the % palmitic acid at sn-2 of total sn-2 positioned fatty acids. The ratio means the % of sn-2 palmitic acid of total palmitic acid {(% of sn-2 palmitic of total sn-2 positioned fatty acids)/3)/(% total palmitic acid)} × 100.

### Example 2

### A Composition of the Invention

A composition comprising a fat base of the invention and additional oils and fats (i.e. Fat blends) that mimic the human breast milk fat composition were prepared as follows:

The fat fraction was produced by blending of fat base with other oils. Oil was mixed together with other components (proteins, carbohydrates, minerals, vitamins and others). The slurry was passed through a pressure homogenizer to get a stable emulsion. Homogenized product was then dried in a spray drier to obtain final product. Other additives may be added to the dry powder to obtain final formulation.

The fat fraction produced by the blending of Fat base with other oils and fats as described above was further blended with other nutrients such as proteins, minerals, vitamins and carbohydrates to yield a food product supplying a subject with the major nutrients also found in human milk. The nutrients and fats were homogenized using pressure homogenization and spray dried to yield a homogenous powder. The powder was further re-dispersed in water (approx. 9 g powder per 60 ml water) to yield a ready-to-feed formula. The fat content of the ready feed was approx. 3.5 g per 100 ml which corresponds to the fat content of human breast milk, which is in the range of 30-40 g/L.

Table 3 shows the fatty acid composition of Fat blend 11 comprising a fat base (30%) mixed/blended with other oils and fats used to create a fat source used in a composition for use in the invention. Table 4 shows details of the ingredients and properties of the composition comprising the fat source of Table 3. Since Fat blends are prepared by blending fat base with other oils, the fatty acids composition of the blends results from the fatty acids composition of both the fat base and of the other oils mixed with the fat base.

TABLE 3

| Fatty acid | % of fatty acids |
|---|---|
| C10:0 | 1.3 |
| C12:0 | 10.3 |
| C14:0 | 4.3 |
| C16:0 | 23.5 |
| C16:0 at sn-2 of total fatty acids | 30.3 |
| at sn-2 | |
| Ratio (%) of sn-2 palmitic acid | 43 |
| of total palmitic acid | |
| C18:0 | 3.2 |
| C18:1 | 39.2 |
| C18:2 | 13.6 |
| C18:3 | 1.7 |
| C20:0 | 0.3 |
| C20:1 | 0.3 |
| C22:0 | 0.2 |

All numbers represent % (w/w), except the ratio which is defined as %. C16:0 represents the total palmitic acid content. C16:0 at the sn-2 represents the % palmitic acid at sn-2 of total sn-2 positioned fatty acids. The ratio means the % of sn-2 palmitic acid of total palmitic acid {(% of sn-2 palmitic of total sn-2 positioned fatty acids)/3)/(% total palmitic acid)}×100.

TABLE 4

| | Per 100 g | Per 100 ml |
|---|---|---|
| Formula | powder | ready to feed |
| Energy (kcal) | 508 | 68 |
| Sodium (mg) | 140 | 18.8 |
| Protein (g) | 11.4 | 1.5 |
| (Lacatalbumin/Casein 60/40) | | |
| Fat (gr) | 26.5 | 3.5 |
| Saturated fat (gr) | 11.3 | 1.49 |
| Linoleic acid (mg) | 5000 | 670 |
| Alpha-linolenic acid (mg) | 530 | 71 |
| Arachidonic acid (mg) | 115 | 15.3 |
| Docosahexaenoic acid (mg) | 108 | 14.4 |
| Cholesterol (mg) | 2 | 0.3 |
| Lactose (gr) | 56 | 7.5 |
| Calcium (mg) | 430 | 57.3 |
| Phosphorus (mg) | 250 | 33.5 |
| Potassium (mg) | 420 | 56.3 |
| Chloride (mg) | 300 | 40.2 |
| Iron (mg) | 5.25 | 0.7 |
| Magnesium (mg) | 50 | 6.7 |
| Zinc (mg) | 3.5 | 0.47 |
| Copper (mcg) | 300 | 40.2 |
| Manganese (mcg) | 45 | 6 |
| Iodine (mcg) | 45 | 6 |
| Taurine (mg) | 45 | 6 |
| Vitamin A I.U. | 1500 | 200 |
| Vitamin D I.U. | 300 | 40.2 |
| Vitamin E (mg) | 10 | 1.3 |
| Vitamin K (mcg) | 45 | 6 |
| Vitamin C (mg) | 60 | 8 |
| Vitamin B₁ (mcg) | 400 | 53 |
| Vitamin B₂ (mcg) | 800 | 127 |
| Vitamin B₆ (mcg) | 375 | 50 |
| Vitamin B₁₂ (mcg) | 1.15 | 0.2 |
| Niacin (mg) | 6 | 0.8 |
| Panthothenic acid (mg) | 3 | 0.4 |
| Folic acid (mcg) | 67 | 9 |
| Biotin (mcg) | 14.3 | 1.9 |
| Choline (mg) | 37.5 | 5 |
| Inositol (mg) | 22.5 | 3 |
| Moisture % | 3 | |

The level of fat and the exact composition can be controlled in order to yield compositions designed to yield formulas designed to mimic the different nutritional needs at different stages and situation in life.

Table 5 shows the fatty acid composition of Fat blend 12 comprising a fat base of the invention blended with other oils and fats used to create a fat source used in a composition for use in the invention.

TABLE 5

| Fatty acid | % from total Fatty acids |
|---|---|
| C8:0 | 1.6 |
| C10:0 | 1.5 |
| C12:0 | 10.6 |
| C14:0 | 3.9 |
| C16:0 | 17.2 |
| C16:0 at sn-2 of total fatty acids at | 26.3 |
| sn-2 | |
| Ratio (%) of sn-2 palmitic acid of | 51 |
| total palmitic acid | |
| C18:0 | 2.4 |
| C18:1 | 41.1 |
| C18:2 | 18.2 |
| C18:3 | 2.2 |
| % Fat base in fat blend | 43 |
| Vegetable Oil | |
| Randomized Coconut oil | 22 |
| Randomized Sunflower | 15 |
| Randomized Rapeseed | 20 |

All numbers represent % (w/w), except the ratio which is defined as %. C16:0 represents the total palmitic acid content. C16:0 at the sn-2 represents the % palmitic acid at sn-2 of total sn-2 positioned fatty acids. The ratio means the % of sn-2 palmitic acid of total palmitic acid {(% of sn-2 palmitic of total sn-2 positioned fatty acids)/3)/(% total palmitic acid)}×100.

### Example 3

### Effect of Fat Base on Rate of Proliferation and Mutagenesis

The effect of the present invention on the rate of proliferation and mutagenesis is examined by an in vitro study using bacterial and mammalian cell culture models. The study includes two groups of oil incubations: one of low palmitic acid content oil (˜8% total palmitic acid, of which about 10% is esterified to sn-2 position of the triglyceride—hereinafter “lower palmitic acid content”) and the second of high sn-2 palmitic acid oil (˜20% total palmitic acid of which ˜50% is esterified to sn-2 position of the triglyceride—hereinafter “higher palmitic acid content”).

The study is performed in three models:

Ames test: this is a biological assay performed in 5 strains of bacteria to assess, the mutagenic potential of chemical compounds. The objective is to test the possible inhibitory effect of fat bases and fat blends of the invention on the incidence of bacterial reverse mutations. In this test each group of bacteria is incubated with one of the oils and the incidence of mutations is examined by Reverse Bacterial Mutation Assay. The study is conducted based on the Ninth Addendum to OECD Guidelines for Testing of Chemicals, Section 4, No. 471, “Bacterial Reverse Mutation Test”, adapted 21 Jul. 1997. This type of study was used in Guzmán A, Evaluation of the genotoxic potential of the natural neurotoxin Tetrodotoxin (TTX) in a battery of in vitro and in vivo genotoxicity assays, Mutation Research, 2007.

Proliferation test: this is a test performed in human peripheral blood mononuclear cells (huPBMCs). The objective of this test is to evaluate the effect of fat bases and fat blends of the invention on the proliferation of the cells. In this test each group of cells is incubated with one of the oils and the rate of proliferations is measured using the Thymidine incorporation assay. This type of study was used in Weissgarten J et. al, Total cell-associated Zn²⁺ and Cu²⁺ and proliferative responsiveness of peripheral blood mononuclear cells from patients on chronic hemodialysis, Metabolism 2001.

Mutagenicity test: this is a test performed in L51 mouse lymphoma cells, which are sensitive to specific mutations. The objective of this test is to study the inhibitory effect of fat bases and fat blends of the invention on the incidence of mammalian cell gene mutations. In this test each group of L51 cells is incubated with one of the oils and the incidence of specific mutations is examined using the Mouse Lymphoma Assay. The study is conducted based on the Ninth Addendum to OECD Guidelines for Testing of Chemicals, Section 4, No. 476, “In Vitro Mammalian Cell Gene Mutation Test”, adapted 21 Jul. 1997. This type of study was used in Whittaker P, Evaluation of commercial kava extracts and kavalactone standards for mutagenicity and toxicity using the mammalian cell gene mutation assay in L578Y mouse lymphoma cells, Food Chem. Toxicol. 2007.

The incidence of mutations in bacteria incubated with the oil with the higher palmitic acid content (high sn-2 palmitic acid) is lower as compared to the bacteria incubated with oil with a lower palmitic acid content as shown by the Reverse Bacterial Mutation Assay.

The proliferation rate of human peripheral blood mononuclear cells (huPBMCs) incubated with the oil with the higher palmitic acid content (high sn-2 palmitic acid) is lower as compared to the bacteria incubated with the oil with the lower palmitic acid content as shown by the Thymidine incorporation assay.

The sensitivity of L51 mouse lymphoma cells incubated with the oil with the higher palmitic acid content (high sn-2 palmitic acid) to the specific mutations tested is lower as compared to the bacteria incubated with the oil with the low palmitic acid content as shown by the Mouse Lymphoma Assay.

### Example 4

### Effect of Fat Base on Rate of Proliferation and Mutagenesis

The efficacy of a Fat blend (see Table 2) on the rate of proliferation and mutagenesis was examined in vitro using bacterial and mammalian cell culture models. The study tested two groups of oils: Fat blend 6 (see Table 2) and a low palmitic acid content oil (LPO) (8% total palmitic acid, of which about 10% is esterified to sn-2 position of the triglyceride) as presented in Table 7.

### Study Design

In order to investigate the potential of the Fat blends of the invention on their ability to reduce gene mutations, three anti-mutagenesis assays were tested:

### 1. Proliferation Assay

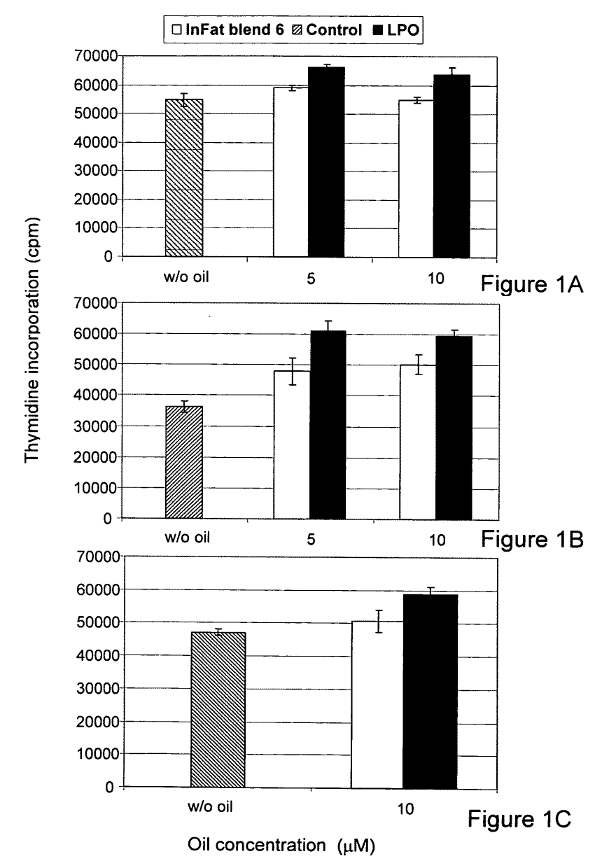

The proliferation assay (Weissgarten J et. al, Total cell-associated Zn²⁺ and Cu²⁺ and proliferative responsiveness of peripheral blood mononuclear cells from patients on chronic hemodialysis, Metabolism 2001) of human peripheral blood mononuclear cells (huPBMCs) was performed to evaluate the effect of Fat blend 6 (Table 2) on the proliferation rate of the cells. huPBMCs cells were freshly prepared from healthy human donor. Separation procedure was performed from enriched leukocytes of full blood based on Ficoll-Hypaque density gradient centrifugation. Cells were immediately thereafter seeded at a density of 10⁵ cells/well in 3×96-well tissue culture plates. huPBMCs cells were incubated with one of two tested oils, i.e. with Fat blend 6 or with LPO. The huPBMCs cells proliferation rate was measured following 5 days of incubation of the tested oils (Fat blend 6 and LPO) with PHA (phytohemagglutinin, Lectin from Phaseolus vulgaris (red kidney bean)), a proliferation activator. The application was conducted by the following routes: (a) the tested oils and the PHA were applied on day 1 of the experiment (FIG. 1A); (b) The tested oils was applied on day 1 and the PHA was applied on day 2 of the experiment, allowing simulation of prevention of mutagenesis rational (FIG. 1B); and (c) PHA was applied on day 1 and the tested oils were applied on day 2 of the experiment, allowing simulation of treatment of mutagenesis rational (FIG. 1C). The proliferation rate was measured using the Thymidine incorporation assay. Following incubation, 22 μL of ³H-Thymidine was added to each well. Cells were harvested 24 hours later. ³H-Thymidine incorporation into the DNA of proliferating cells was measured with a β-counter.

### 2. Mouse Lymphoma Assay (MLA) Assay

The MLA assay evaluates the mutagenic potential of the tested oils based on quantitation of forward mutations at the tk locus of L5178Y in mouse lymphoma cells. The mutagenicity of the test agents is indicated by the increase in the number of mutants after treatment. The tk enzyme is responsible for incorporating exogenous thymidine via a salvage pathway, into the cell in the form of thymidine monophosphate. Analogues of thymidine, such as trifluorothymidine (TFT) can also be phosphorylated by tk enzyme, which leads to cell toxicity. Forward mutation at this locus results in a loss of tk activity and subsequent resistance to TFT, which is used as the selective agent to kill wild type cells. Thus the tk mutants are not killed by TFT and are able to survive due to their ability to synthesize purines de novo.

The objective of the MLA assay was to study the inhibitory effect of Fat blend 6 (Table 2) on the incidence of mammalian cell gene mutations. Each group of L5178Y cells was incubated with one of the tested oils, i.e. with Fat blend 6 or with oil containing low palmitic acid (LPO), and the incidence of specific mutations was examined using the MLA assay. The study experiments were performed with a constant concentration of reference mutagen. Additionally, several concentrations of the test items were used. Positive and negative controls were included in the experiment.

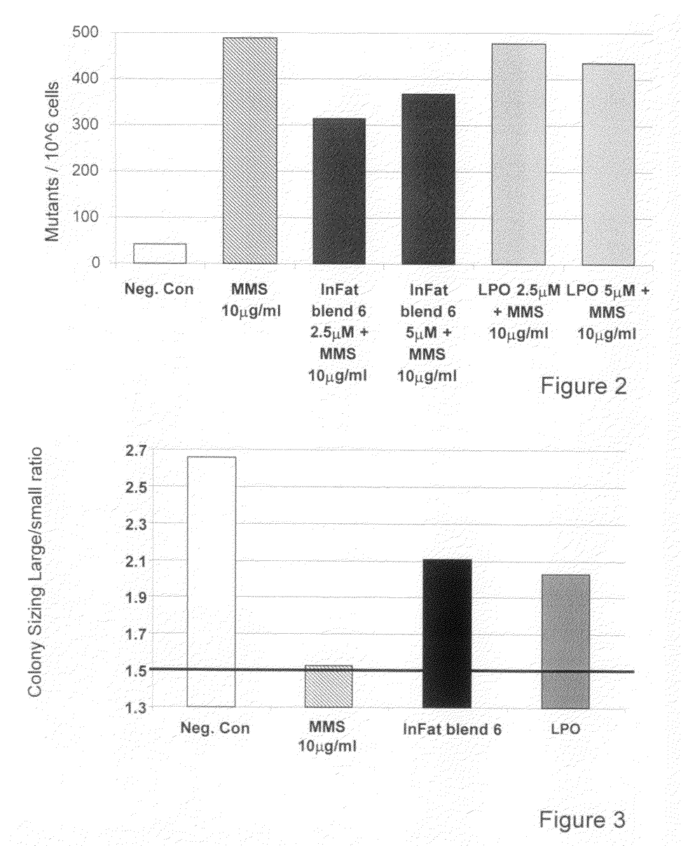

Study procedure: 1×10⁷ cells/culture (80 cm² flasks) were exposed to several concentrations of tested oil in the presence of the mutagen (10 μg/ml MMS) in parallel to the negative and positive controls (Table 6, FIG. 2). Cells were incubated with the tested oil and mutagen for 3-4 hours to induce and increase the efficiency of anti-mutagenesis effect. Following the incubation, the mutagen was removed by centrifugation (400 g, 5 min) and the cells were washed twice. Subsequently the cells were re-suspended in growth medium for an expression period of 4 days. Following expression period, cultures were seeded in selective medium (containing TFT). Cells from each experimental group were seeded in four 96-well plates at a density of 2000 cells/well in selective medium. After an incubation period of 11 days at 37±1° C., 5±0.5% CO₂ and 95±5% humidified atmosphere, the number of colony containing/empty wells was scored using microscope. The colonies size and/or morphology were also characterized. The inhibitory effect of the 2 tested oils was evaluated using the following criteria: (a) a decrease of Mutation Frequencies in the presence of tested oil related to the positive control. (b) Decreased occurrence of small colonies (slow growth colonies) indicated by a high large/small colonies ratio was an indication for inhibitory potential clastogenic effects and/or chromosomal aberrations.

TABLE 6
Experimental cultures plan
Treatment

Negative Control
Positive control (10 μg/ml MMS)
MMS (10 μg/ml) and Fat blend 6 (2.5 μM, 5 μM or 10 μM)
MMS (10 μg/ml) and LPO tested oil (2.5 μM, 5 μM or 10 μM)

The study was conducted based on the Ninth Addendum to OECD Guidelines for Testing of Chemicals, Section 4, No. 476, “In Vitro Mammalian Cell Gene Mutation Test”, adapted 21 Jul. 1997. This type of study was used in Whittaker P, Evaluation of commercial kava extracts and kavalactone standards for mutagenicity and toxicity using the mammalian cell gene mutation assay in L5178Y mouse lymphoma cells, Food Chem. Toxicol. 2007.

### 3. Modified Reverse Mutation Assay (Ames Assay)

Ames assay was performed in the Salmonella typhimurium bacteria and its objective was to assess the mutagenic potential of chemical compounds. Bacterial reverse mutation assays use amino acid requiring strains of Salmonella typhimurium to detect point mutations, which involve substitution, addition or deletion of one or a few DNA base pairs. The principle of these bacterial reversion assays is that they detect mutations which functionally reverse mutations present in the tester strains and restore the capability to synthesize essential amino acids. The Salmonella typhimurium histidine (his) reversion system measures his⁻ his⁺ reversions. Point mutations are the cause of many human genetic diseases and there is substantial evidence that somatic cell point mutations in oncogene suppressor genes are involved in cancer. This study tested the possible inhibitory effect of Fat blend 6 (Table 2) on the incidence of bacterial reverse mutations following incubation with Fat blend 6 or with an oil containing low palmitic acid (LPO) (Table 7).

The reference mutagen (MMS) was mixed with the tested oils at each dose level, with or without metabolic activation (S9) mix. The bacteria most commonly used in these assays do not possess the enzyme system which, in mammalians, is known to convert pro-mutagens into DNA damaging metabolites. In order to overcome this major drawback an exogenous metabolic system is added in the form of a mammalian microsome enzyme mixture. This mammalian microsome enzyme mixture is S9 liver microsomal fraction which was prepared from rats liver.

Study procedure: samples of the tester strains were grown by culturing for 12 hr at 38.5° C. in nutrient broth to the late exponential or early stationary phase of growth (10⁹ cells/ml). The bacteria were exposed to eight different concentrations of the tested oils concurrent with defined constant concentration of reference mutagen. The mutagen (0.2 μl/plate of methyl methane sulfonate (MMS)) was mixed with one strain of Salmonella typhimurium bacteria, TA 102, strain and was plated on minimal agar plates that do not contain any histidine. The concentration range of the tested oils covered three logarithmic decades. The assay was performed with the following concentrations: 0.014, 0.041, 0.123, 0.37, 1.11, 3.33, 10 and 30 nmol/plate.

The exposure of the tested strains to the tested oils or the control solution was performed using incorporation methods, i.e. the bacteria suspension was mixed in a test tube with the tested oil, the mutagen, either with or without S9, and poured over the surface of a minimal agar plate. After solidification the plates were inverted and incubated at 37° C. for at least 48 h in the dark. Following the incubation period revertant colonies were counted. To validate the test, reference mutagens were tested in parallel to the test item. Strain specific positive controls were included in the assay, which demonstrated the effective performance of the assay. The mutation factor was calculated by dividing the mean value of the revertant counts through the mean values of the solvent control.

The study was conducted based on the internationally accepted guidelines and recommendations: Ninth Addendum to OECD Guidelines for Testing of Chemicals, Section 4, No. 471, “Bacterial Reverse Mutation Test”, adapted 21 Jul. 1997. EEC Directive 2000/32 L 136 “Mutagenicity-Reverse Mutation Test Bacteria”, Annex 4D, B 13/14 dated May 19, 2000, EPA Health Effects Test Guidelines, OPPTS 870.5100, Bacterial Reverse Mutation Test EPA 712-C-98-247, August 1998. This type of study was used in Guzmán A, Evaluation of the genotoxic potential of the natural neurotoxin Tetrodotoxin (TTX) in a battery of in vitro and in vivo genotoxicity assays, Mutation Research, 2007.

TABLE 7
Fatty acids composition of oils (% of total fatty acids)
before final assays concentrations dilutions

| fatty acid (as % from | high palmitic acid content oil | low palmitic acid |
|---|---|---|
| total Fatty acid) | (Fat blend 6) | content oil |
| C8 | 0.6 | 1.1 |
| C10 | 0.6 | 1.0 |
| C12 | 8.7 | 14.05 |
| C14 | 3.5 | 4.9 |
| C16 | 21 | 8.3 |
| C16:0 at sn-2 of total | 31.8 | 29.5 |
| fatty acids at sn-2 | | |
| Ratio (%) of sn-2 | 50.5 | 11.9 |
| palmitic acid of total | | |
| palmitic acid | | |
| C16:1 | 0.00 | 0.1 |
| C18 | 2.6 | 2.6 |
| C18:1 | 44.4 | 47.6 |
| C18:2 | 16.4 | 18.1 |
| C18:3 | 1.5 | 1.6 |
| C20 | 0.2 | 0.3 |
| C20:1 | 0.3 | 0.4 |

All numbers represent % (w/w), except the ratio which is defined as %. C16:0 represents the total palmitic acid content. C16:0 at the sn-2 represents the % palmitic acid at sn-2 of total sn-2 positioned fatty acids. The ratio means the % of sn-2 palmitic acid of total palmitic acid {(% of sn-2 palmitic of total sn-2 positioned fatty acids)/3)/(% total palmitic acid)}×100.

### 1. Proliferation Test Results

When the tested oils and the PHA were applied to the cells on day 1 of the experiment, huPBMCs cells incubated with 5 μM and 10 μM of Fat blend 6 demonstrated a proliferation rate which was significantly lower than cells treated with similar concentrations of LPO (P=0.005 and 0.002 respectively) (FIG. 1A). There were no significant differences between proliferation of huPBMCs cells which were not treated with oils (control) and cells which were treated with Fat blend 6 as shown by the Thymidine incorporation assay.

A similar result was shown when the tested oils were applied before the PHA. The proliferation rate of huPBMCs cells incubated with the 5 μM and 10 μM of Fat blend 6 was significantly lower than cells treated with similar contractions of LPO (P=0.021 and 0.017 respectively) (FIG. 1B).

The proliferation rate of untreated huPBMCs cells (control) was significantly lower than cells treated both with 5 μM and 10 μM Fat blend 6 (P=0.02 and 0.002 respectively).

When PHA was applied a day before the tested oils, the proliferation rate of huPBMCs cells was significantly lower when the cells were treated with 10 μM of Fat blend 6 when compared to cells treated with the same concentration of LPO (P=0.037) (FIG. 1C).

There was no significant difference between proliferation of untreated huPBMCs cells (control) and cells which were treated with Fat blend 6.

Conclusions: A significant increase in proliferation of huPBMCs cells treated with lower palmitic acid content (LPO) was demonstrated when compared to the control cells. A decrease in proliferation of huPBMCs cells treated with Fat blend 6 was demonstrated as compared to cells treated with LPO.

Elevated mutagenesis and decreased DNA repair at a transgene are associated with proliferation. In addition, uncontrolled and often rapid proliferation of cells can lead to benign tumors. When comparing to low palmitic acid oils, the decreased rate of proliferation in the presence of Fat blend 6 (oil containing high palmitic acid at sn-2 position) proves the ability of the fat blends and fat bases of invention to protect the cells against proliferation or reproduction of plasma cells that might be a result of mutagenesis.

### 2. MLA Test Results

### Anti Mutagenesis Results

Toxicity of the tested oils on the cell cultures was tested. The relative suspension growth (RSG) of cultures incubated with the tested oils and with the mutagen MMS (10 μg/ml), compared to the negative control group, had similar cytotoxic effects as those incubated with the MMS (10 μg/ml) positive control group (77.5% and 65.8% respectively). The toxicity results of the tested oils, in addition to absolute cloning efficiency tests, proved to be sufficient and demonstrated the acceptability of the assay, and the anti mutagenesis study could be further tested.

The mutation frequencies of the positive control group (10 μg/ml MMS) were tested and showed distinct and biological relevant effects in a dose response manner.

The mutation analysis data are presented in Table 8 and FIG. 2. The mean of cultures per 96 wells plate (Table 8) express the number of cultures in which mutation had occurred and therefore the cultures survived the selection medium. Negative control, in which only spontaneous mutations had occurred, showed the lowest number of cultures. The number of cultures and the number of mutants per 10̂6 cells (Table 8, FIG. 2) in cells incubated with 2.5 μM and 5 μM Fat blend 6 (39 and 41 cultures respectively, 315, 367 mutants per 10̂6 cells respectively) decreased when compared to the positive control (10 μg/ml MMS) (46 cultures, 489 mutants per 10̂6 cells) (Table 8, FIG. 2). However, the number of cultures was similar to the positive control when the cells were incubated with 2.5 μM and 5 μM LPO oil (47.5 and 42.8 cultures respectively, 476, 435 mutants per 10̂6 cells respectively). In a similar manner, the mutation factors (Table 8) decreased when incubated with Fat blend 6 and did not change when incubated with LPO, when compared to the positive control; The mutation factor of cells incubated with MMS (positive control) decreased from 12 to 7 and 9 when incubated also with 2.5 μM and 5 μM Fat blend 6 respectively. The mutation factor of cells incubated with 2.5 μM and 5 μM LPO oil remained 11 and 10 respectively, similar to the positive control (Table 8, FIG. 2). The percentage change in mutants in cells incubated with 2.5 μM Fat blend 6 when compared to the cells treated with MMS only (positive control) decreased to 64%. Thus, cells exposed to Fat blend 6 and to 10 μg/ml MMS indicate a reduction of mutant frequency when compared to the positive control group (10 μg/ml MMS) and to cells incubated with the LPO oil.

TABLE 8
Mutagenicity data

| | Oil | Mean of | | | % change |
|---|---|---|---|---|---|
| | concen- | cultures | | | in mutants |
| | tration | per 96 | Mutants/ | Mutation | compare to |
| | (μM) | wells plate | 10\hat{ }6 cells | factor | MMS treat |
| Negative | | 9.5 | 42 | 1 | |
| Control | | | | | |
| Positive | | 46.0 | 489 | 12 | 100 |
| Control- | | | | | |
| MMS | | | | | |
| Fat | 2.5 | 39.0 | 315 | 7 | 64 |
| blend 6 | 5 | 41.0 | 367 | 9 | 75 |
| LPO | 2.5 | 47.5 | 476 | 11 | 98 |
| | 5 | 42.8 | 435 | 10 | 89 |

Colonies Size and/or Morphology Results

Decreased occurrence of small colonies (slow growth colonies), indicated by a high large/small colonies ratio, is an indication for inhibitory potential of clastogenic effects and/or chromosomal aberrations. A large/small colony sizing ratio lower than coefficient of 1.5 (marked line in FIG. 3) is considered clastogenic. The quotient of large/small colonies of the negative control was found to be 2.66 (Table 9, FIG. 3) which is considered non-clastogenic. The quotient of the positive controls test group (10 μg/ml MMS) was with coefficient of 1.53 which is considered clastogenic. The quotient of large/small colonies incubated with Fat blend 6 was 2.11 and the quotient of large/small colonies incubated with LPO oil was 2.03, both colony sizing ratios which are considered non-clastogenic. The conclusion is that Fat blend 6 and LPO oil at the highest dose (10 μM) showed a trend of non-clastogenic effects as compared with the controls of the assay.

TABLE 9
Colonies sizing

| | Colony Sizing Large/small |
|---|---|
| Negative Control | 2.66 |
| MMS | 1.53 |
| Fat blend 6 | 2.11 |
| LPO | 2.03 |

The size of the colonies was characterized as followed: small colonies approximately <¼ of well diameter and large colonies approximately >¼ of well diameter.

### 3. Ames Study

The toxicity effects of the tested oils on the bacteria strains were examined. No toxic effects were noted in the tester strain used up to the highest dose evaluated of the tested oils, with and without metabolic activation. In addition, the reference mutagens induced a distinct increase of revertant colonies indicating the validity of the experiments.

Based on the experimental variation observed within the historical laboratory control data, reductions in the number of revertants below a mutation factor of 0.7 are considered biological relevant. The measured results are presented in Table 10. The reductions in the number of revertants of TA 102 tested strain incubated with Fat blend 6 decreased to mutation factor of 0.2 at a concentration of 0.014 nmole/plate, decreased to a mutation factor of 0.5 at a concentration of 0.41 nmole/plate and decreased to a mutation factor of 0.5 at concentration of 10 nmole/plate (Table 10). The effects of reductions in the number of revertants of tested strain exposed to LPO were much less significant when compared to Fat blend 6. Although no dose response relationship was found, the evidence of the decreased number of revertants following exposure to Fat blend 6 indicates that when compared to LPO, Fat blend 6 has a protective effect against mutagenesis.

TABLE 10
The reductions in the number of revertants.

| | Tester | Tested oil dose | Mutation | Mutation |
|---|---|---|---|---|
| | strain | (nmole) | factor (−S9) | factor (+S9) |
| Fat | TA 102 | 0.014 | 0.2 | |
| blend 6 | | 0.041 | 0.5 | |
| | | 0.123 | 0.7 | |
| | | 0.37 | 0.9 | |
| | | 10 | 0.5 | |
| | | 30 | 0.8 | |
| LPO | TA 102 | 0.014 | 0.6 | 0.6 |
| | | 0.041 | 0.8 | 0.8 |
| | | 10 | 0.7 | 0.6 |

1. A method of treating or preventing a condition in a subject having, or having an increased risk of incidences of mutagenesis, carcinogenesis and proliferative disease or disorder comprising administering to the subject a lipid composition comprising at least one triglyceride of the following formula I: 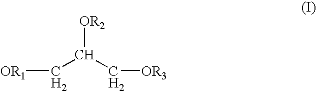
wherein R₁, R₂ and R₃ may be identical or different and are each independently selected from H or an acyl group, wherein said acyl group is selected from a group consisting of saturated, mono-unsaturated and poly-unsaturated fatty acid residues, and wherein the total palmitic acid residue content is from about 15% to about 55% of the total fatty acid residues in the composition.

2. The method according to claim 1, wherein R₂ is a saturated fatty acid residue.

3. The method according to claim 2, wherein the saturated fatty residue is selected from C₁₄-C₁₈ saturated fatty acid residues.

4. The method according to claim 3, wherein the saturated fatty acid is a palmitic acid residue.

5. The method according to claim 1, wherein the total palmitic acid residue content is from about 15% to about 40% of the total fatty acid residues in the composition

6. The method according to claim 5, wherein the total palmitic acid residue content is from about 15% to about 33% of the total fatty acid residues in the composition

7. The method according to claim 1, wherein R₁ and R₃ are both H.

8. The method according to claim 1, wherein at least 13% of the total fatty acids at the sn-2 position of the triglyceride backbone are palmitic acid residues.

9. The method according to claim 8, wherein at least 15% of the total fatty acids at the sn-2 position of the triglyceride backbone are palmitic acid residues.

10. The method according to claim 9, wherein at least 18% of the total fatty acids at the sn-2 position of the triglyceride backbone are palmitic acid residues.

11. The method according to claim 10, wherein at least 22% of the total fatty acids at the sn-2 position of the triglyceride backbone are palmitic acid residues.

12. The method according to claim 1, wherein at least 30% of the total palmitic acid residues are bonded at the sn-2 position of the triglyceride backbone.

13. The method according to claim 12, wherein at least 33% of the total palmitic acid residues in the composition are bonded at the sn-2 position of the triglyceride backbone.

14. The method according to claim 13, wherein at least 38% of the total palmitic acid residues are bonded at the sn-2 position of the triglyceride backbone.

15. The method according to claim 14, wherein at least 40% of the total palmitic acid residues are bonded at the sn-2 position of the triglyceride backbone.

16. The method according to claim 1, wherein R₁ and R₃ are unsaturated fatty acid residues.

17. The method according to claim 16, wherein at least 50% of the total fatty acid residue at the sn-1 and sn-3 positions of the triglyceride backbone are unsaturated.

18. The method according to claims 17, wherein at least 70% of the total fatty acid residue at the sn-1 and sn-3 positions of the triglyceride backbone are unsaturated.

19. The method according to claim 16, wherein the unsaturated fatty acid residue is selected from the group consisting of oleic acid, linoleic acid, linolenic acid and gadoleic acid.

20. The method according to claim 19, wherein at least 35% of the unsaturated fatty acid residue at the sn-1 and sn-3 positions are oleic acid residues.

21. The method according to claim 20, wherein at least 40% of the unsaturated fatty acid residues at the sn-1 and sn-3 positions are oleic acid residues.

22. The method according to claim 19, wherein at least 4% of said unsaturated fatty acid residues at the sn-1 and sn-3 positions are linoleic acid residues.

23. The method according to claim 22, wherein at least 6% of said unsaturated fatty acid residues at the sn-1 and sn-3 positions are linoleic acid residues.

24. The method according to claim 1, wherein the lipid consists of
0-10% C8:0 fatty acids out of the total fatty acids;
0-10% C10:0 fatty acids out of the total fatty acids;
0-22% C12:0 fatty acids out of the total fatty acids;
0-15% C14:0 fatty acids out of the total fatty acids;
15-55% C16:0 fatty acids out of the total fatty acids; wherein at least 30% at sn-2 position;
1-7% C18:0 fatty acids out of the total fatty acids;
20-75% C18:1 fatty acids out of the total fatty acids;
2-40% C18:2 fatty acids out of the total fatty acids;
0-8% C18:3 fatty acids out of the total fatty acids; and
other fatty acids in an amount of less than 8% of the total fatty acids.

25. The method according to claim 22, wherein the lipid consists of
5-15% C12:0 fatty acids out of the total fatty acids;
2-10% C14:0 fatty acids out of the total fatty acids;
17-25% C16:0 fatty acids out of the total fatty acids; wherein at least 40% at sn-2 position;
2-5% C18:0 fatty acids out of the total fatty acids;
28-45% C18:1 fatty acids out of the total fatty acids;
5-20% C18:2 fatty acids out of the total fatty acids;
1-3% C18:3 fatty acids out of the total fatty acids; and
other fatty acids in an amount of less than 5% of the total fatty acids.

26. The method according to claim 1, wherein the lipid composition is prepared from a natural, synthetic or semi-synthetic source.

27. The method according to claim 26, wherein the natural source is any one of plant, animal or microorganism source.

28. The method according to claim 26, wherein the production of the lipid composition comprises enzymatic catalysis.

29. The method according to claim 1, wherein the lipid composition is a nutritional, pharmaceutical or nutraceutical composition or a functional food

30. The method according to claim 29, wherein the pharmaceutical or nutraceutical composition is in a dosage delivery form.

31. The use method according to claim 1, wherein the functional food is selected from the group consisting of dairy product, ice-cream, biscuit, soy product, bakery, pastry and bread, sauce, soup, prepared food, frozen food, condiment, confectionary, oils and fat, margarine, spread, filling, cereal, instant product, drinks and shake, infant food, toddler food, bar, snack, candy and chocolate product.

32. The use method according to claim 29, wherein the pharmaceutical composition further comprises at least one pharmaceutically active agent.

33. The method according to claim 1, wherein the mutagenesis, carcinogenesis and proliferative disease or disorder is selected from the group consisting of cancer, autoimmune disorders, anti-inflammatory diseases, cutaneous lymphoproliferative diseases, and rheumatic diseases.

34. The method according to claim 33, wherein cancer is selected from the group consisting of leukemia, melanoma, breast cancer, pancreatic cancer, lung cancer, prostate cancer, colorectal and/or colon cancer, hepatocellular carcinoma, lymphoma, sarcoma, mesothelioma, brain cancer, germinoma, choriocarcinoma, renal cancer, thyroid cancer, head and neck cancer, endometrial cancer, cervical cancer, bladder cancer, stomach cancer, glioma, anaplastic astrocytoma, and glioblastoma multiforme.

35. The method according to claim 29, wherein the nutritional composition is selected from the group consisting of human milk fat substitute, infant formula, dairy product, ice-cream, biscuit, soy product, bakery, pastry and bread, sauce, soup, prepared food, frozen food, condiment, confectionary, oils and fat, margarine, spread, filling, cereal, instant product, infant food, toddler food, bar, snack, candy and chocolate product.

36. (canceled)

37. The method according to claim 1, further comprising administering to the subject at least one additional drug.

38. The method according to claim 1, further comprising subjecting the subject to at least one additional anti-proliferative treatment.

39. The method according to claim 1, wherein the subject is an infant.

40. The method according to claim 39, wherein the infant is a preterm or term infant.

41. The method according to claim 37, wherein the mutagenesis, carcinogenesis and proliferative disease or disorder is selected from the group consisting of cancer, autoimmune disorders, anti-inflammatory diseases, cutaneous lymphoproliferative diseases, and rheumatic diseases.

42. The method according to claim 41, wherein the cancer is selected from the group consisting of leukemia, melanoma, breast cancer, pancreatic cancer, lung cancer, prostate cancer, colorectal and/or colon cancer, hepatocellular carcinoma, lymphoma, sarcoma, mesothelioma, brain cancer, germinoma, choriocarcinoma, renal cancer, thyroid cancer, head and neck cancer, endometrial cancer, cervical cancer, bladder cancer, stomach cancer, glioma, anaplastic astrocytoma, and glioblastoma multiforme.
